(19) United States
(12) Patent Application Publication
(10) Pub. No.: US 2025/0352373 A1
(43) Pub. Date: Nov. 20, 2025

(54) STENT WITH HYBRID CONSTRUCTION

(71) Applicant: BOSTON SCIENTIFIC SCIMED, INC., Maple Grove, MN (US)

(72) Inventors: LOUIS MCNERN, Donegal (IE); DANIEL TUCK, Galway (IE); MARTYN G. FOLAN, Galway (IE); GARY GILMARTIN, Foxford (IE)

(73) Assignee: BOSTON SCIENTIFIC SCIMED, INC., Maple Grove, MN (US)

(21) Appl. No.: 19/212,081

(22) Filed: May 19, 2025

Related U.S. Application Data
(60) Provisional application No. 63/649,730, filed on May 20, 2024.

(51) Int. Cl. A61F 2/90

(52) U.S. Cl. CPC A61F 2/90; A61F 2230/0091; A61F 2310/00389

(57) ABSTRACT

An illustrative stent may include an expandable framework with a proximal end, a distal end, and a length extending therebetween. The expandable framework of the stent may include a single filament forming one or more undulating pattern sections and one or more knitted sections. The expandable framework of the stent may further include one or more of a coating and a covering.

### CROSS-REFERENCE TO RELATED APPLICATIONS

The application claims the benefit of U.S. Provisional Patent Application Ser. No. 63/649,730, filed on May 20, 2024, the disclosure of which is incorporated herein by reference.

### TECHNICAL FIELD

The present disclosure pertains to medical devices, methods for manufacturing medical devices, and uses thereof. More particularly, the present disclosure pertains to a stent for implantation in a body lumen, and associated methods.

### BACKGROUND

A wide variety of intracorporeal medical devices have been developed for medical use, for example, surgical and/or intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods.

### SUMMARY

This disclosure provides design, material, manufacturing method, and use alternatives for medical devices. An example medical device may include a stent.

In a first example, a stent may include an expandable framework. The expandable framework may include an expandable framework body including a proximal and a distal end. The expandable framework may further include one or more coated regions which include a first filament woven in a first pattern. The expandable framework may further include one or more covered regions which include a second filament woven in a second pattern. The first pattern may be a different pattern than the second pattern, while the one or more coated regions may be adjacent to the one or more covered regions.

Alternatively or additionally to any of the examples above, the first pattern may be an undulating pattern, a helical pattern or a combination of both.

Alternatively or additionally to any of the examples above, the second pattern may be a knitted pattern.

Alternatively or additionally to any of the examples above, the one or more coated regions may include a polymeric coating.

Alternatively or additionally to any of the examples above, the one or more covered regions may be covered by a cylindrical sleeve.

Alternatively or additionally to any of the examples above, the one or more coated regions may include at least two spaced apart coated regions.

Alternatively or additionally to any of the examples above, one or more of the expandable framework body, one or more coated regions, and one or more covered regions may be heat set to be biased toward an expanded configuration of the expandable framework body.

Alternatively or additionally to any of the examples above, the first filament and the second filament may be the same filament.

In another example, a stent may include an expandable framework. The expandable framework may include an expandable framework body including a proximal end and a distal end. The expandable framework body may be formed of a single filament. The expandable framework body may also include one or more first regions which include the filament woven in a first undulating pattern. The expandable framework body may also include one or more second regions which include the filament woven in a knitted pattern. One or more first regions may be adjacent to at least one of the one or more second regions.

Alternatively or additionally to any of the examples above, the one or more first regions may include a polymeric coating.

Alternatively or additionally to any of the examples above, the one or more second regions may be covered by a cylindrical sleeve.

Alternatively or additionally to any of the examples above, the one or more first regions may include at least two spaced apart first regions.

Alternatively or additionally to any of the examples above, the one or more second regions may include at least two spaced apart second regions.

In another example, a method of forming a stent is contemplated. In this and other examples, a method may include: receiving a woven expandable framework including one or more knitted-filament regions and one or more undulating-filament regions over a mandrel; applying a coating to the one or more undulating-filament regions; and applying a circumferential sleeve around the one or more knitted-filament regions. The step of applying the coating may be performed separately from the step of applying the circumferential sleeve.

Alternatively or additionally to any of the examples above, the step of applying a coating to the one or more undulating-filament regions may further include applying a spray or dip coating.

Alternatively or additionally to any of the examples above, the step of applying a circumferential sleeve around the one or more knitted-filament region may further include applying one or more of a sheath, a sleeve and a wrap.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments in connection with the accompanying drawings, in which:

FIG. 1 is a side view of an illustrative stent;

FIG. 1A is an enlarged view of an undulating pattern of the stent of FIG. 1;

FIG. 1B is an enlarged view of a knitted pattern of the stent of FIG. 1;

FIG. 2 is a side view of an illustrative stent;

FIG. 3 is a side view of an illustrative stent;

FIG. 4 is a side view of an illustrative stent with coating and covering applied; and

FIG. 5 is a schematic view of a mandrel for use in coating a stent.

While the disclosure is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used herein, the term “distal” refers to the end farthest away from the medical professional when introducing a medical device into a patient, while the term “proximal” refers to the end closest to the medical professional when introducing a medical device into a patient.

The term “patient,” as used herein, is synonymous with the term “subject” and generally refers to any animal under the care of a medical professional, as that term is defined herein, with particular reference to (i) humans (under the care of a doctor, nurse, or medical assistant or volunteer) and (ii) non-human animals, such as non-human mammals (under the care of a veterinarian or other veterinary professional, assistant, or volunteer).

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

In some instances, it may be desirable to provide an endoluminal implant, or stent, that can deliver and/or regain luminal patency in a patient with a biliary obstruction or other medical condition. Such stents may be used in patients experiencing bile duct blockage and are deployed to maintain patency in the bile duct and allow proper passage of bile into the small bowel. Some stents have a woven or knitted configuration to provide good radial strength with minimal foreshortening which may be desirable in biliary applications as well as some esophageal and post-bariatric surgery applications. However, some knitted stent designs may be difficult to constrain, especially into a coaxial delivery system and thus may be delivered using a system which may not offer a method of recapture. What may be desirable is an alternative knitted stent that incorporates alternating patterns of knitted filaments and undulating filaments that is capable of delivery via a coaxial delivery system while having similar radial forces, conformability and foreshortening as previous knitted stent configurations. While the embodiments disclosed herein are discussed with reference to biliary stents, it is contemplated that the stents described herein may be used and sized for use in other locations such as, but not limited to: bodily tissue, bodily organs, vascular lumens, non-vascular lumens and combinations thereof, such as, but not limited to, in the coronary or peripheral vasculature, trachea, bronchi, colon, small intestine, esophagus, urinary tract, prostate, brain, stomach and the like.

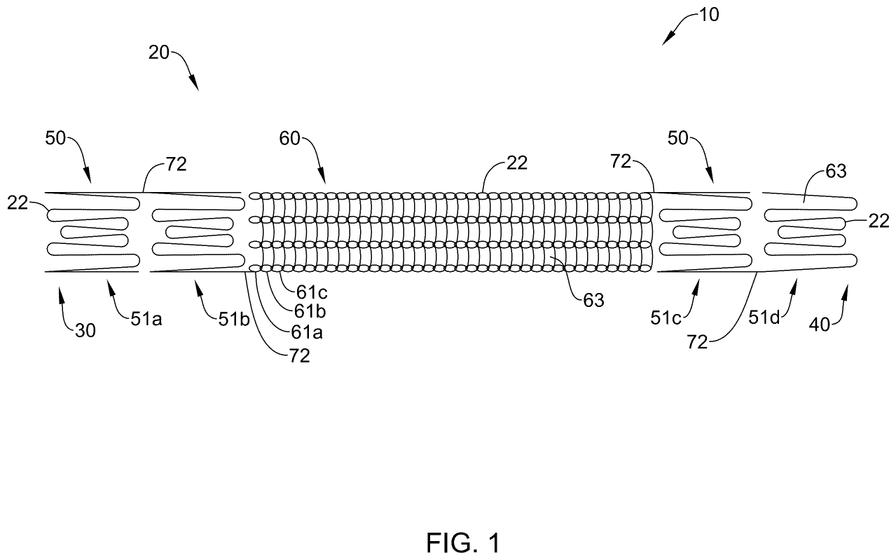

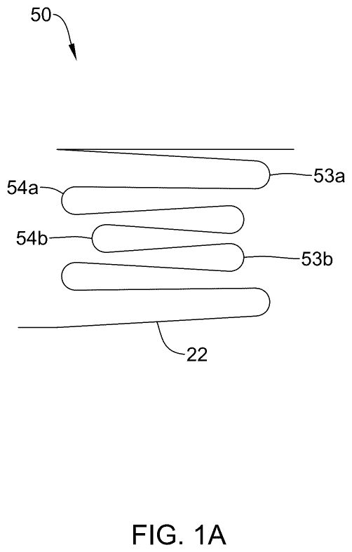

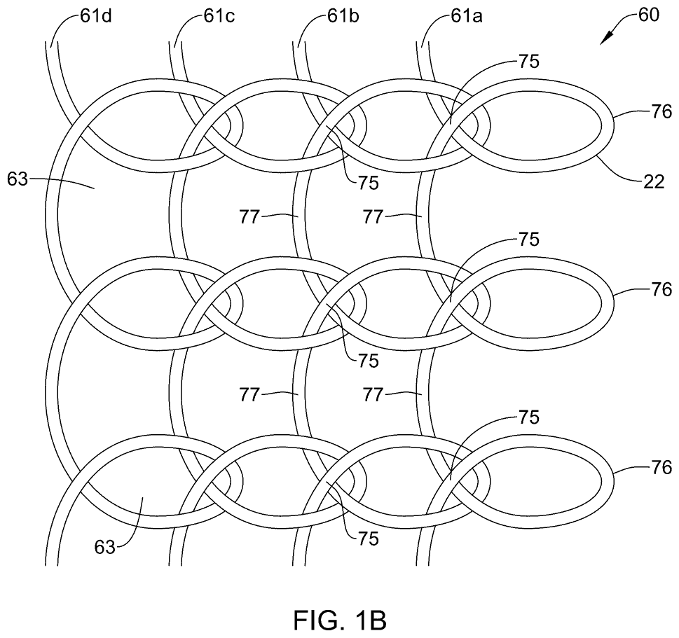

FIG. 1 illustrates a side view of an illustrative endoluminal implant, such as, but not limited to, a stent 10. In some instances, the stent 10 may comprise an expandable tubular framework 20 which extends from a first, proximal end 30 to a second, distal end 40 and defines a lumen extending therethrough. The tubular framework 20 may include one or more sections of a filament 22 woven in an undulating pattern 50 and one or more sections of the filament 22 woven in a knitted pattern 60. As shown in FIG. 1, a first end region (e.g., proximal end region extending to the first, proximal end 30) may be a first region including the filament 22 formed in an undulating pattern 50 and a second end region (e.g., distal end region extending to the second, distal end 40) may be a second region including the filament 22 formed in an undulating pattern 50. The medial region, e.g., the region extending between the proximal end region and the distal end region, may be a third region including the filament 22 formed in a knitted pattern 60, such as a twisted knitted pattern, as described herein.

The advantages of constructing a stent 10 with alternating sections of undulating pattern 50 and knitted pattern 60 as depicted in FIG. 1 (and further described herein), are myriad. Advantages of this innovative design include, but are not limited to greater conformability of the stent 10 through torturous anatomy of a patient or subject, a greater reduction in foreshortening of the stent 10, and/or a greater reduction of kinking of the expandable framework 20 of the stent 10 after deployment. To improve luminal or vessel patency further, the stent 10 may be heat set by a process described further herein.

Accordingly, a single filament 22 may be woven into one or both of the undulating pattern 50 and the knitted pattern 60. In other words, the filament 22 woven into the undulating pattern 50 and the knitted pattern 60 may be the same filament. It can be appreciated a single filament 22 can be designated for different sections, such as an undulating pattern section and a knitted pattern section. It can further be appreciated that these sections may possess different structural properties than the other sections. For instance, the section of filament 22 woven into the undulating pattern may have a higher flexural modulus, a higher torsional modulus, a higher Young's modulus, a higher shear modulus, a higher bulk modulus, a greater hardness, a higher resistance to radial force, a higher resistance to tensile force, a greater resistance to pressure, a greater resistance to hemodynamic pressure, a greater resistance to fluid pressure, an increased Poisson's ratio, a greater resistance to temperature, a greater density, a greater weight, a larger surface area, a larger volume, and/or a larger surface-area-to-volume-ratio than the section of the filament 22 woven into the knitted pattern, or vice-versa.

The undulating pattern 50 may be a helical pattern, a serpentine pattern, a wavy pattern, an oscillating pattern, a sinusoidal pattern, a waveform, a step pattern, a spiked pattern or the like. Alternatively or additionally, knitted pattern 60 may be a twisted knit stitch pattern (see U.S. Pat. No. 11,266,517, incorporated herein by reference in its entirety), an open knit stitch pattern (see U.S. Pat. No. 5,876,445, incorporated herein by reference in its entirety), or the like. As shown in FIG. 1, the first, proximal end region and/or the second, distal end region may include multiple cylindrical segments formed of the undulating pattern 50. For instance, the first, proximal end region may include a first cylindrical segment 51a of the undulating pattern 50 and a second cylindrical segment 51b of the undulating pattern 50 with a connecting portion 72 of the filament 22 interconnecting the first cylindrical segment 51a and the second cylindrical segment 51b. Likewise, the second, distal end region may include a third cylindrical segment 51c of the undulating pattern 50 and a fourth cylindrical segment 51d of the undulating pattern 50 with a connecting portion 72 of the filament 22 interconnecting the third cylindrical segment 51c and the fourth cylindrical segment 51d.

FIG. 1A is an enlarged view of an undulating pattern 50 of the stent 10. Throughout the undulating pattern 50, the filament 22 may include a plurality of bends forming a plurality of peaks and opposing valleys. In some instances, the circumferential region having the undulating pattern 50 may include one or more peaks that are not axially aligned with one or more additional peaks in the circumferential region. For example, as shown in FIG. 1A, the undulating pattern 50 may include one ore more, or a plurality of first peaks 53a that extend axially further than one or more, or a plurality of second peaks 53b. Additionally or alternatively, the undulating pattern 50 may include one or more, or a plurality of first valleys 54a that extend axially further than one or more, or a plurality of second valleys 54b. Other arrangements and configurations of the peaks and valleys of the undulating pattern 50 are also contemplated.

When the knitted pattern 60 is formed from a single filament 22 forming a twisted knit stitch pattern, the filament 22 may be manipulated (e.g., knitted) into a plurality of circumferential rows each having a plurality of interconnected or intermeshing closed loops. Three adjacent circumferential rows of the twisted knitted pattern are labeled in FIG. 1, which includes a first circumferential row 61a, a second circumferential row 61b adjacent to and interconnected with the first circumferential row 61a, and a third circumferential row 61c adjacent to and interconnected with the second circumferential row 61b. The knitted sections 60 of stent 10 may include as many circumferential rows as required to form knitted sections 60 of the tubular scaffold 20 of the stent 10 having the desired length. As described above, the closed loops may be loosely knit and include interconnecting intermediate rung portions interconnecting adjacent closed loops. It should be understood that as the knitted sections 60 of the stent 10 are formed from a single filament 22, the circumferential rows of the knitted section 60 may not be distinct and separate rows but instead extending helically around the tubular scaffold 20 in a continuous fashion. In some instances, a closed loop may be generally aligned with, or suspended from, a closed loop of the preceding row in a direction generally parallel to a longitudinal axis of the stent 10 (for example, circumferentially aligned along a length of the stent 10). Thus, the closed loop in one row may be suspended from the closed loop in a preceding row.

To form the knitted sections 60 of stent 10, an end region (distal end portion) of the filament 22 is passed over an intermediate rung portion of a preceding row. The end region of the filament 22 may then be wrapped behind the closed loop in a direction opposite to the general direction of the overall knit. The end region of the filament 22 may then be passed over a rung portion on an opposing side of the closed loop (relative to the rung portion) before being crossed over itself to complete the closed loop. Thus, each closed loop may be formed of a segment of the filament 22 in which the filament 22 crosses over itself to close the loop. The reverse configuration is also contemplated in which the loop passes behind the rung portions and over the closed loop of a preceding row. The closed loops may generally take the form of a twisted knit stitch where each individual loop is twisted having end regions of the closed loop that cross over one another and extend in opposite directions.

FIG. 1B is an enlarged view of a knitted pattern of the stent of FIG. 1. The filament 22 can been seen forming multiple circumferential rows, including a first circumferential row 61a, a second circumferential row 61b, a third circumferential row 61c, and a fourth circumferential row 61d. Each circumferential row includes closed loop portions 76 interconnected by an intermediate rung portion 77 extending between adjacent closed loop portions 76. The closed loops 76 may be closed by having the filament 22 cross over itself at a cross-over point 75 at a base of each closed loop 76. The closed loops 76 of each succeeding row may extend around the cross-over point 75 of the closed loops 76 of a previous row.

It is contemplated that the knitted pattern(s) 60 of the stent 10 and the undulating pattern(s) 50 of the stent 10 may be formed with a single filament 22 construction defining open cells 63 defined between adjacent portions of the single filament 22 forming the tubular wall or scaffold of the stent 10. The open cells 63 may each define an opening from an outer surface of the tubular wall or scaffold 20 to an inner surface of the tubular wall or scaffold 20 (e.g., through a thickness thereof) that is free from the filament 22.

As described herein, the stent 10 may be formed of a single filament 22 extending throughout the undulating section(s) 50 and the knitted section(s) 60. As such, the one or more undulating sections 50 may be directly adjacent a knitted section 60 and/or one or more knitted sections 60 may be directly adjacent an undulating section 50, with the filament 22 transitioning from a knitted section 60 to an undulating section 50, or vice versa, via a connecting portion 72 (e.g., connector) of the filament 22. Thus, the filament 22 may form a single connector 72 between a knitted section 60 and an adjacent undulating section 50. In other words, a length of the filament 22 (i.e., connecting portion 72) extending between a knitted section 60 and an adjacent undulating section 50 may be the sole connection of the tubular scaffold 20 between the knitted section 60 and the adjacent undulating section 50.

The stent 10 may be expandable from a first radially collapsed configuration (not explicitly shown) to a second radially expanded configuration. In some instances the length of the stent 10 (measured from the proximal end 30 to the distal end 40) in its radially expanded configuration may be substantially the same as the length of the stent 10 in its radially collapsed configuration. For example, the length of the stent 10 in its radially expanded configuration may be at least 90% or more, or 95% or more of its length in the radially collapsed configuration, in some instances. In some cases, the stent 10 may be deployed to a configuration between the collapsed configuration and a fully expanded configuration. The stent 10 may be structured to extend across a biliary duct and to apply a radially outward pressure to the lumen of the duct to open the lumen, maintain patency within the lumen and allow for the passage of obstructions, fluids, food, air, etc.

It is contemplated that the stent 10 can be made from a number of different materials such as, but not limited to, metals, metal alloys, shape memory alloys and/or polymers, as desired, enabling the stent 10 to be expanded into shape when accurately positioned within the body. In some instances, the material may be selected to enable the stent 10 to be removed with relative ease as well. For example, the filament 22 of the stent 10 can be formed from alloys such as, but not limited to, Nitinol and Elgiloy®. Depending on the material selected for construction, the stent 10 may be self-expanding (i.e., configured to automatically radially expand when unconstrained). In some embodiments, the filament 22 used to make the stent 10, which may be a composite filament, for example, may have an outer shell made of Nitinol and having a platinum core. It is further contemplated the stent 10 may be formed from polymers including, but not limited to, polyethylene terephthalate (PET). In some embodiments, the stent 10 may be self-expanding while in other embodiments, the stent 10 may be expanded by an expansion device (such as, but not limited to a balloon inserted within the lumen of the stent 10). As used herein the term “self-expanding” refers to the tendency of the stent 10 to return to a preprogrammed or heat set diameter when unrestrained from an external biasing force (for example, but not limited to a delivery catheter or sheath). In some instances, the stent 10 may include a one-way valve, such as an elastomeric slit valve or duck bill valve, positioned within the lumen thereof to prevent retrograde flow of gastrointestinal fluids.

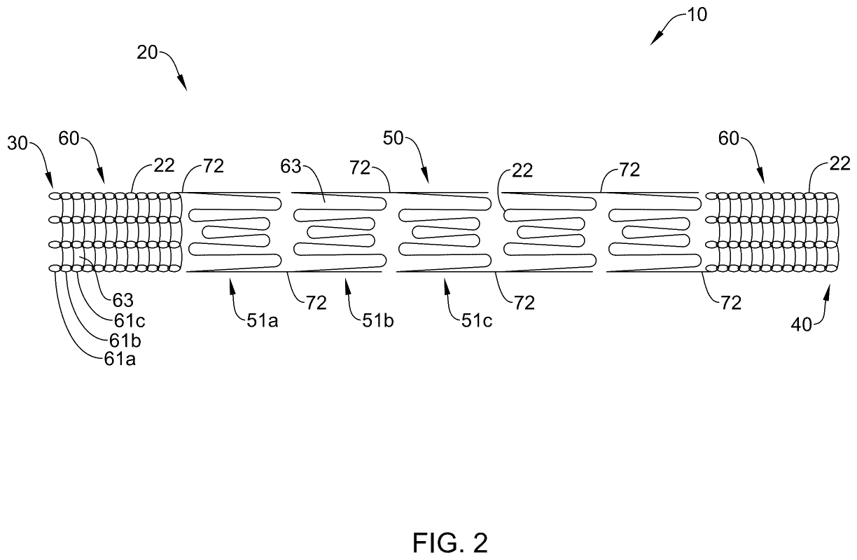

FIG. 2 illustrates a side view of an example stent 10 of the present disclosure. The stent may include an expandable tubular framework 20 which extends from a first, proximal end 30 to a second, distal end 40 and defines a lumen extending therethrough. The tubular framework 20 may include alternating sections of a filament 22 woven in an undulating pattern 50 and a knitted pattern 60. In other words, one section of the expandable framework 20 of the stent 10 may include a filament 22 woven in an undulating pattern 50, and another section of the expandable framework 20 of the stent 10 may include the filament 22 woven in a knitted pattern 60.

As shown in FIG. 2, a first end region (e.g., proximal end region extending to the first, proximal end 30) may be a first region including the filament 22 formed in a knitted pattern 60, such as a twisted knitted pattern, as described herein, and a second end region (e.g., distal end region extending to the second, distal end 40) may be a second region including the filament 22 formed in a knitted pattern 60, such as a twisted knitted pattern, as described herein. Three adjacent circumferential rows of the twisted knitted pattern are labeled in FIG. 2, which includes a first circumferential row 61a, a second circumferential row 61b adjacent to and interconnected with the first circumferential row 61a, and a third circumferential row 61c adjacent to and interconnected with the second circumferential row 61b.

The medial region, e.g., the region extending between the proximal end region and the distal end region, may be a third region including the filament 22 formed in an undulating pattern 50, such as a twisted knitted pattern, as described herein. As shown in FIG. 2, the medial region may include multiple cylindrical segments formed of the undulating pattern 50. For instance, a first cylindrical segment 51a of the undulating pattern 50 may be interconnected to a second cylindrical segment 51b of the undulating pattern 50 with a connecting portion 72 of the filament 22, the second cylindrical segment 51b may be interconnected to a third cylindrical segment 51c, and so on.

This pattern may repeat, alternate, irregularly repeat, irregularly alternate, or ascribe to any desired pattern. For instance, the expandable framework 20 of the stent 10 may include alternating sections of the filament 22 (i.e., a single filament) woven into an undulating pattern 50 and a knitted pattern 60. Alternatively or additionally, the sections of undulating pattern 50 and knitted pattern 60 may be spaced apart with a connecting portion 72 (e.g., connector) of the filament 22 extending therebetween. The sections of undulating pattern 50 and knitted pattern 60 may be spaced apart by any suitable distance.

In some instances, the sections of undulating pattern 50 and knitted pattern 60 may be spaced apart by 0.5 mm or more, 0.10 mm or more, or 0.2 mm or more. In some instances, the sections of undulating pattern 50 and knitted pattern 60 may be spaced apart by 10 mm or less, 5 mm or less, or 2 mm or less. In some non-limiting examples, the sections of undulating pattern 50 and knitted pattern 60 may be spaced apart by 0.75 mm, by 1 mm, by 1.15 mm, by 1.25 mm, by 1.4 mm, by 1.5 mm, by 1.75 mm, by 2 mm, by 2.25 mm, by 2.5 mm, by 2.75 mm, by 3 mm, by 3.25 mm, by 3.5 mm, by 3.75 mm, by 4 mm, by 4.25 mm, by 4.5 mm, by 4.75 mm, by 5 mm, by 5.25 mm, by 5.5 mm, by 5.75 mm, by 6 mm, by 7 mm, by 8 mm, by 9 mm, by 10 mm, by 11 mm, by 12 mm, by 15 mm, by 20 mm, or any suitable or equivalent range or value of measurement desired.

In some instances, each section of the filament 22 woven in an undulating pattern 50 may be of greater length (i.e., longer) than each other section of the filament 22 woven in a knitted pattern 60. In other words, the length from the proximal end of a section of the filament 22 woven in an undulating pattern 50 to the distal end of the same section may be of greater length (i.e., longer) than any corresponding section of the filament 22 woven in a knitted pattern 60. One such configuration is shown in FIG. 2. Alternatively, in some instances, each section of the filament 22 woven in a knitted pattern 60 may be of greater length (i.e., longer) than each other section of the filament 22 woven in an undulating pattern 50. In other words, the length from the proximal end of a section of the filament 22 woven in a knitted pattern 60 to the distal end of the same section may be of greater length (i.e., longer) than any corresponding section of the filament 22 woven in an undulating pattern 50. One such configuration is shown in FIG. 1.

In some instances, each section of the filament 22 woven in an undulating pattern 50 may have a greater outer diameter in the radially expanded configuration than each other section of the filament 22 woven in a knitted pattern 60. In some instances, each section of the filament 22 woven in a knitted pattern 60 may have a greater outer diameter in the radially expanded configuration than each other section of the filament 22 woven in an undulating pattern 50. In some instances, the diameter of the filament 22 may vary between the regions or sections of the stent 10 having an undulating pattern 50 and the regions or sections of the stent having a knitted pattern 60. For instance, in the section(s) or region(s) having an undulating pattern 50, the filament 22 may be of greater diameter than in any corresponding section(s) or region(s) of the stent 10 having a knitted pattern 60. In other instances, in the section(s) or region(s) having a knitted pattern 60, the filament 22 may be of greater diameter than in any corresponding section(s) or region(s) of the stent 10 having an undulating pattern 50. In some instances, the section(s) or region(s) in which the filament 22 has a smaller diameter may be subjected to a chemical etching process, for example, to reduce the diameter of the filament 22 in the corresponding section(s) or region(s).

In some instances, the lengths of each of the undulating pattern 50 and knitted pattern 60 sections may be varying with respect to each of the other undulating pattern 50 and knitted pattern 60 sections. In other words, myriad lengths are contemplated for any and all undulating pattern 50 and knitted pattern 60 sections, and those lengths may be the same, differing, varying, related by a ratio, related by a pattern, or otherwise desired. It can be appreciated that one or more undulating sections 50 may be longer than one or more knitted sections 60 while also being shorter than one or more other knitted sections 60, and vice-versa.

In some instances, the outer diameters of each of the undulating pattern 50 and knitted pattern 60 sections may be varying with respect to each of the other undulating pattern 50 and knitted pattern 60 sections. It can be appreciated that one or more undulating sections 50 may possess a greater outer diameter than one or more knitted sections 60 while also possessing a smaller diameter than one or more other knitted sections 60, and vice-versa.

It is contemplated that the knitted pattern(s) 60 of the stent 10 and the undulating pattern(s) 50 of the stent 10 may be formed with a single filament 22 construction defining open cells 63 defined between adjacent portions of the single filament 22 forming the tubular wall or scaffold of the stent 10. The open cells 63 may each define an opening from an outer surface of the tubular wall or scaffold 20 to an inner surface of the tubular wall or scaffold 20 (e.g., through a thickness thereof) that is free from the filament 22.

As described herein, the stent 10 may be formed of a single filament 22 extending throughout the undulating section(s) 50 and the knitted section(s) 60. As such, the one or more undulating sections 50 may be directly adjacent a knitted section 60 and/or one or more knitted sections 60 may be directly adjacent an undulating section 50, with the filament 22 transitioning from a knitted section 60 to an undulating section 50, or vice versa, via a connecting portion 72 (e.g., connector) of the filament 22. Thus, the filament 22 may form a single connector 72 between a knitted section 60 and an adjacent undulating section 50. In other words, a length of the filament 22 (i.e., connecting portion 72) extending between a knitted section 60 and an adjacent undulating section 50 may be the sole connection of the tubular scaffold 20 between the knitted section 60 and the adjacent undulating section 50.

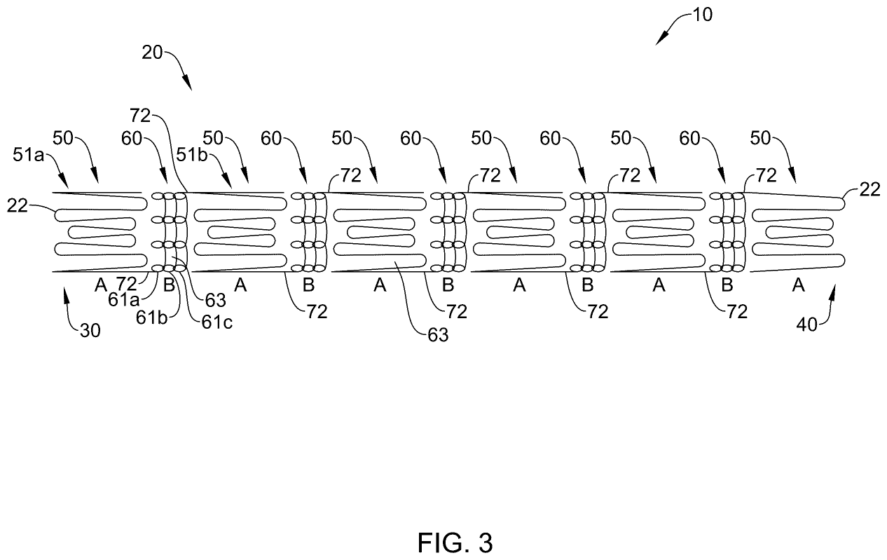

FIG. 3 illustrates a side view of an illustrative stent 10 including an expandable tubular framework 20 extending from a first, proximal end 30 to a second, distal end 40, and defining a lumen extending therethrough. Depicted along the expandable framework 20 of the stent 10 are repeating sections of a single filament 22 woven in an undulating pattern 50 and a knitted pattern 60. These alternating sections are further depicted in FIG. 3 as A and B, respectively. In other words, the letter ‘A’ denotes a section of the single filament 22 woven in an undulating pattern 50, and the letter ‘B’ denotes a section of the single filament 22 woven in a knitted pattern 60. As shown in FIG. 3 and contemplated herein, sections A and B are in a spaced apart configuration. As further shown in FIG. 3, and contemplated herein, sections A may be of greater length (i.e., longer) than sections B. However, as contemplated in other examples, sections B may of greater length (i.e., longer) than sections A. In yet other examples, a first section A (the section A nearest the proximal end 30 of the expandable framework 20) may be longer than a first section B (the section B nearest the proximal end 30 of the expandable framework 20) but may be shorter than a second section B (the section B second-nearest the proximal end 30 of the expandable framework 20). Myriad combinations and permutations of the above are contemplated, including but not limited to a stent 10 comprising an expandable framework 20 which may include alternating sections of a single filament 22 woven in an undulating pattern 50 and a knitted pattern 60, whereby the alternating sections may be of varying lengths, such that a first section A (seen as the A nearest proximal end 30 in FIG. 3), is shorter than a first section B (seen as the B nearest the proximal end 30 in FIG. 3), shorter than a second section B (seen as the second B from the proximal end 30 of stent 10), but longer than a third section B (seen as the third B from the proximal end 30 of the stent 10). Myriad other combinations and permutations are also contemplated, including but limited to a ratio between section lengths. In other words, in some instances, the ratio of the length of the undulating sections 50 (e.g., each section A (undulating pattern 50)) to the length of the knitted sections 60 (e.g., each section B (knitted pattern 60)) may be 1:1 or more, 2:1 or more, 3:1 or more, 4:1 or more, or 5:1 or more. In some instances, the ratio of the length of the knitted sections 60 (e.g., each section B (knitted pattern 60) to the length of the undulating sections 50 (e.g., each section A (undulating pattern 50)) may be 1:1 or more, 2:1 or more, 3:1 or more, 4:1 or more, or 5:1 or more. In some instances, each section A (undulating pattern 50) may be twice the length of each section B (knitted pattern 60), such that section A is of greater length than section B by a 2:1 ratio. Myriad other ratios are contemplated, including but not limited to 1:1 (i.e., the length of each section A equals the length of each section B), 3:1 (i.e., the length of each section A is three-times greater than the length of each section B), 1:3 (i.e., the length of each section B is three-times greater than the length of each section A), or other arrangement.

Further contemplated herein are exemplary pattern routines for the design of stent 10. The expandable framework 20 of stent 10 may include a pattern of the filament 22 (or filaments) woven in an undulating pattern 50 (denoted by A) and the filament 22 (or filaments) woven in a knitted pattern 60 (denoted by B). Exemplary pattern routines include, but are not limited to A-B-A-B-A-B (i.e., a pattern of undulating-knitted-undulating-knitted-undulating-knitted sections), A-A-B-A-A (i.e., a pattern of undulating-undulating-knitted-undulating-undulating sections), B-A-B-A-B (i.e., a pattern of knitted-undulating-knitted-undulating-knitted sections), B-B-A-B-B (i.e., a pattern of knitted-knitted-undulating-knitted-knitted sections), B-A-B-B-A-B (i.e., a pattern of knitted-undulating-knitted-knitted-undulating-knitted sections). All other combinations and permutations of the aforementioned are contemplated, including but not limited to adding extra sections to the expandable framework 20 of stent 10.

It is contemplated that the knitted pattern(s) 60 of the stent 10 and the undulating pattern(s) 50 of the stent 10 may be formed with a single filament 22 construction defining open cells 63 defined between adjacent portions of the single filament 22 forming the tubular wall or scaffold of the stent 10. The open cells 63 may each define an opening from an outer surface of the tubular wall or scaffold 20 to an inner surface of the tubular wall or scaffold 20 (e.g., through a thickness thereof) that is free from the filament 22.

As described herein, the stent 10 may be formed of a single filament 22 extending throughout the undulating section(s) 50 and the knitted section(s) 60. As such, the one or more undulating sections 50 may be directly adjacent a knitted section 60 and/or one or more knitted sections 60 may be directly adjacent an undulating section 50, with the filament 22 transitioning from a knitted section 60 to an undulating section 50, or vice versa, via a connecting portion 72 (e.g., connector) of the filament 22. Thus, the filament 22 may form a single connector 72 between a knitted section 60 and an adjacent undulating section 50. In other words, a length of the filament 22 (i.e., connecting portion 72) extending between a knitted section 60 and an adjacent undulating section 50 may be the sole connection of the tubular scaffold 20 between the knitted section 60 and the adjacent undulating section 50. As shown in FIG. 3, a first twisted knitted pattern 60 (including a first circumferential row 61a, a second circumferential row 61b adjacent to and interconnected with the first circumferential row 61a, and a third circumferential row 61c adjacent to and interconnected with the second circumferential row 61b) may be positioned between a first cylindrical segment 51a of the undulating pattern 50 and a second cylindrical segment 51b of the undulating pattern 50. This may be repeated along the length of the stent 10.

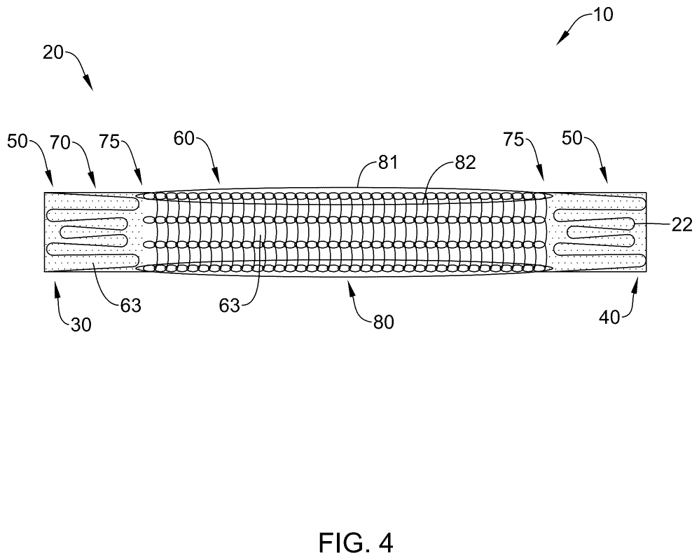

FIG. 4 illustrates another example stent 10 of the present disclosure. Stent 10 is depicted schematically. A coating 70 is applied to the undulating pattern sections 50 and a covering 80 is applied to the knitted pattern section 60. The covering 80 is distinct from the coating 70, as described herein. Any of the above described stent configurations can similarly include a combination of a coating applied to the undulating pattern section(s) 50 and a covering 80 applied to the knitted pattern section(s) 60.

The coating 70 may be any desired coating, including but not limited to a spray coating or a dip coating, such as a polymeric coating. The polymeric coating may be a polyurethane coating, a silicone coating, or the like.

In some instances, the filament 22 of the expandable framework 20 may be encapsulated by the coating 70 such that the coating 70 spans the open cells defined between adjacent portions of the filament 22 throughout the undulating pattern sections 50 of the stent 10. For instance, an inner and/or outer surface of the expandable framework 20 of the stent 10 may be entirely, substantially, or partially, covered with the coating 70 throughout the undulating pattern sections 50 of the stent 10.

The covering 80 may be a sheath or sleeve disposed along the knitted pattern section(s) 60 of the tubular scaffold 20. The covering 80 may be formed of a polymeric material, such as polyurethane, silicone, polyester, polyether block amide, or the like.

The covering 80 may include an inner layer 82 extending along an interior of the knitted region 60 of the tubular framework 20 and an outer layer 81 extending along an exterior of the knitted region 60 of the tubular framework 20, such that the knitted region 60 of the tubular framework 20 is arranged between the inner layer 82 and the outer layer 81 of the covering 80. In such instances, the filament 22 of the knitted region 60 may be positioned between the inner layer 82 and the outer layer 81 of the covering 80 without the covering 80 being affixed to or secured to the knitted region 60, permitting the filament 22 of the knitted region 60 to move relative to the covering 80. Furthermore, an outer surface of the inner layer 82 may be spaced apart from an inner surface of the outer layer 81 along substantially an entire length of the covering 80, other than the terminal ends of the covering 80. This may permit the filament 22 of the knitted region 60 to readily shift as the tubular framework 20 is radially expanded and/or radially collapsed. In other words, the filament 22 forming the closed loops and interconnecting rungs may move within the void space formed between the inner layer 82 and the outer layer 81 of the covering 80 during radial expansion and/or radial contraction of the tubular framework 20. Thus, the size of the rung portions and the size of the closed loop portions of the knitted region 60 may change during radial expansion and/or radial contraction of the tubular framework 20.

The proximal end of the covering 80 may be affixed or secured to the coating 70 disposed on the proximal undulating section 50 at a first circumferential attachment point 62 and the distal end of the covering 80 may be affixed or secured to the coating 70 disposed on the distal undulating section 50 at a second circumferential attachment point 64. Thus, the covering 80 may span throughout the medial knitted section 60 and be affixed to the coating 70 adjacent each undulating section 50. In this and other examples, the proximal and/or distal ends of the covering 80 may be affixed or secured to the coating 70 by any of the following processes, including but not limited to: heat bonding, thermal bonding, adhesive bonding, chemical bonding or the like.

Alternatively or additionally, the inner layer 82 and the outer layer 81 of the covering 80 may be bonded circumferentially around the stent 10 and to the coating 70 by any bonding process disclosed herein or any combination or permutation of bonding processes disclosed herein. Multiple inner layers 82 and outer layers 81 are also contemplated and may be implemented as desired. In non-limiting examples, the covering 80 of the present disclosure may include at least two inner layers 82 and at least one outer layer 81. In other non-limiting examples, the covering 80 of the present disclosure may include at least two outer layers 81 and at least one inner layer 82. In yet other non-limiting examples, the covering 80 of the present disclosure may include at least three outer layers 81 and at least two inner layers 82, or vice-versa. Any combination or permutation of inner layers 82 and outer layers 81 are also contemplated and may be implemented as desired.

Alternatively or additionally, the covering 80 may extend along the entire inner luminal area of the knitted region 60. In other words, the interior circumferential surface of the knitted region 60 may be entirely covered by a covering 80. In this and other examples, the inner layer 82 of the covering 80 may include an additional covering or covering layer attached to the outside of the inner layer 82. Alternatively or additionally, an additional covering may be applied to the outer layer 81 of the covering 80. In other non-limiting examples, the covering 80 may extend along a part or portion of the luminal area of the knitted region 60. In other words, the interior circumferential surface of the knitted region 60 may be partially covered by a covering 80. In this and other examples, the inner layer 82 of the covering 80 may include an additional covering or covering layer attached to the outside of the inner layer 82. Alternatively or additionally, an additional covering may be applied to the outer layer 81 of the covering 80.

Alternatively or additionally to any of the examples disclosed herein, the covering 80 may extend along the entire outer circumferential surface area of the knitted region 60. In other words, the exterior circumferential surface of the knitted region 60 may be entirely covered by a covering 80. In other non-limiting examples, the covering 80 may extend along a part or portion of the exterior circumferential surface area of the knitted region 60. In other words, the exterior circumferential surface of the knitted region 60 may be partially covered by a covering 80.

The advantages to providing a combination of a covering 80 and coating 70 to stent 10 are myriad. This innovative design improves the ability of the stent 10 to maintain patency in a patient vessel or lumen by reducing the incidence of food impaction or an obstruction entering the expandable framework 20 of stent 10. This innovative design further offers greater flexibility and conformability of the stent 10 as it is deployed and maintained within tortuous patient anatomy (i.e., the bends of lumens and vessels). For instance, applying a covering 80 to the knitted region 60 allows the filament 22 (or filaments) to adjust and shift (while maintaining a tubular form within the covering 80) within the covering 80 so as to better accommodate tortuous patient anatomy. Further, this innovative design resists common problems with endoprostheses and implants by resisting kinking and foreshortening of the stent 10.

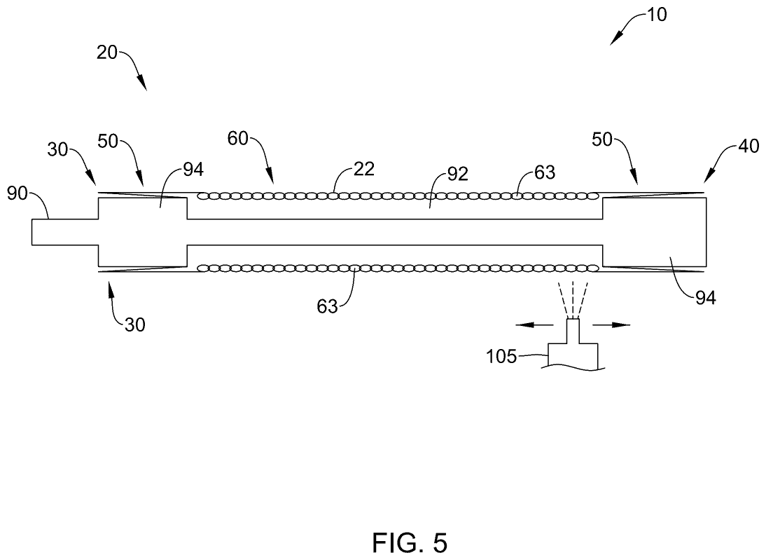

FIG. 5 illustrates a side view of a process of applying the coating 70 to the tubular scaffold 20 of the stent 10 with the tubular scaffold 20 disposed on a coating mandrel 90. The coating mandrel 90 may include a reduced diameter 92 medial region between first and second enlarged diameter end regions 94. The tubular scaffold 20 may be disposed on the coating mandrel 90 with the first and second undulating pattern regions 50 surrounding the first and second enlarged diameter end regions 94, and the knitted pattern region 60 surrounding the medial reduced diameter region 92. The undulating regions 50 may be positioned adjacent to and in contact with the outer surface of the enlarged diameter end regions 94, while the knitted pattern region 60 is radially spaced apart from the outer surface of the reduced diameter region 92.

The coating 70 may then be spray coated over the expandable scaffold 20, such as with a spray nozzle depositing the coating 70 along the length of the expandable scaffold 20 and around the circumference of the expandable scaffold 20. The spray nozzle may translate the full length of stent 10 while rotating stent 10 and mandrel 90 together, for example. It can be appreciated that spray may pass through the open cells 63 of the tubular scaffold 20 throughout the knitted region 60 since the surface of the mandrel 90 is spaced away from the knitted region 60 of the tubular scaffold 20, whereas the spray may be deposited on the undulating regions 50 with the outer surface of the mandrel 90 there beneath (e.g., substantially flush with the inner surface of the tubular scaffold 20 along the undulating regions 50), allowing the coating 70 to form on the undulating regions 50. The layer of material applied to the undulating regions 50 may form the coating 70 once dried and/or cured, whereas the knitted region 60 may remain devoid of a coating thereon.

Thereafter, a covering 80 may be applied to the knitted region 60. For example, a first, or inner polymeric layer (e.g., a first circumferential sleeve) may be disposed in the lumen of the tubular scaffold 20 and extend along an inner surface of the knitted region 60 and/or an outer polymeric layer (e.g., a second, outer circumferential sleeve) may be disposed around the exterior of the tubular scaffold 20 and extend along an outer surface of the knitted region 60.

The first, proximal end of the covering 80 may be affixed or secured to the coating 70 disposed on the first, proximal undulating section 50 at a first circumferential attachment point 75 (see FIG. 4) and the second, distal end of the covering 80 may be affixed or secured to the coating 70 disposed on the second, distal undulating section 50 at a second circumferential attachment point 75 (see FIG. 4). For example, the covering 80 (the inner and/or outer layer) may be thermally bonded, adhesively bonded, chemically bonded, or otherwise affixed to the coating 70. Thus, the covering 80 may span throughout the medial knitted section 60 and be affixed to the coating 70 adjacent each undulating section 50.

The advantages to providing one or more of a covering 80 and coating 70 to stent 10 through the methods described herein are myriad. This innovative approach and design improves the ability of the stent 10 to maintain patency in a patient vessel or lumen by reducing the incidence of food impaction or an obstruction entering the expandable framework 20 of stent 10. This innovative design further offers greater flexibility and conformability of the stent 10 as it is deployed and maintained within tortuous patient anatomy (i.e., the bends of lumens and vessels). Further, this innovative design resists common problems with endoprostheses and implants by resisting kinking and foreshortening of the stent 10.

It is contemplated that the stent 10 of the methods described herein can be made from a number of different materials through the methods disclosed herein, such as, but not limited to, metals, metal alloys, shape memory alloys and/or polymers, as desired, enabling the stent 10 to be expanded into shape when accurately positioned within the body. In some instances, the material may be selected to enable the stent 10 to be removed with relative ease as well. For example, the stent 10 can be formed from alloys such as, but not limited to, Nitinol and Elgiloy®. Depending on the material selected for construction, the stent 10 may be self-expanding (e.g., configured to automatically radially expand when unconstrained). In some embodiments, a composite filament may be used to make the stent 10, which may, for example, having an outer shell made of Nitinol and a platinum core. It is further contemplated the filament forming the tubular scaffold of the stent 10 may be formed from polymers including, but not limited to, polyethylene terephthalate (PET).

The stents, delivery systems, and the various components of the methods contemplated thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic Nitinol; other nickel alloys such as nickel-chromium-molybdenum alloys, nickel-copper alloys, nickel-cobalt-chromium-molybdenum alloys, nickel-molybdenum alloys, other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys; platinum enriched stainless steel; titanium; combinations thereof; and the like; or any other suitable material.

Some examples of suitable polymers for the stents or delivery systems for use in the methods contemplated herein may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

In at least some embodiments, portions or all of the stents or delivery systems for use with stent 10 may also be doped with, made of, or otherwise include a radiopaque material. Radiopaque materials are generally understood to be materials which are opaque to RF energy in the wavelength range spanning x-ray to gamma-ray (at thicknesses of <0.005″). These materials are capable of producing a relatively dark image on a fluoroscopy screen relative to the light image that non-radiopaque materials such as tissue produce. This relatively bright image aids the user of the stents or delivery systems in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stents or delivery systems to achieve the same result.

In some instances, a degree of Magnetic Resonance Imaging (MRI) compatibility is imparted into the apparatus and/or other elements disclosed herein. For example, the apparatus, and/or components or portions thereof, may be made of a material that does not substantially distort the image and create substantial artifacts (e.g., gaps in the image). Certain ferromagnetic materials, for example, may not be suitable because they may create artifacts in an MRI image. The apparatus, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the apparatus and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope is, of course, defined in the language in which the appended claims are expressed.

What is claimed is:

1. A stent comprising:
an expandable framework, the expandable framework comprising:
an expandable framework body including a proximal end and a distal end;
one or more coated regions comprising a first filament woven in a first pattern;
one or more covered regions comprising a second filament woven in a second pattern;
wherein the first pattern is a different pattern than the second pattern; and
wherein the one or more coated regions are adjacent to the one or more covered regions.

2. The stent of claim 1, wherein the first pattern is an undulating pattern, a helical pattern or a combination of both.

3. The stent of claim 1, wherein the second pattern is a knitted pattern.

4. The stent of claim 1, wherein the one or more coated regions comprise a silicone coating.

5. The stent of claim 1, wherein the one or more covered regions is covered by a member selected from the group comprising a sheath, a sleeve and a wrap.

6. The stent of claim 1, wherein the one or more coated regions comprise at least two spaced apart coated regions.

7. The stent of claim 1, wherein the one or more covered regions comprise at least two spaced apart covered regions.

8. The stent of claim 1, wherein one or more of the expandable framework body, one or more coated regions, and one or more covered regions is heat set to be biased toward an expanded configuration of the expandable framework body.

9. The stent of claim 1, wherein the first filament and the second filament are the same filament.

10. A stent comprising:
an expandable framework, the expandable framework comprising:
an expandable framework body including a proximal end and a distal end, the expandable framework body formed of a single filament;
wherein the expandable framework body includes one or more first regions comprising the filament woven in a first undulating pattern and one or more second regions comprising the filament woven in a knitted pattern;
wherein each of the one or more first regions is adjacent to at least one of the one or more second regions.

11. The stent of claim 10, wherein the one or more first regions comprise a polymeric coating.

12. The stent of claim 10, wherein the one or more second regions is covered by a cylindrical sleeve.

13. The stent of claim 10, wherein the one or more first regions comprise at least two spaced apart first regions.

14. The stent of claim 10, wherein the one or more second regions comprise at least two spaced apart second regions.

15. The stent of claim 10, wherein one or more of the expandable framework body, one or more first regions, and one or more second regions is heat set to be biased toward an expanded configuration of the expandable framework body.

16. The stent of claim 10, wherein each of the one or more first regions is longer than each of the one or more second regions.

17. The stent of claim 10, wherein each of the one or more second regions is longer than each of the one or more first regions.

18. A method of forming a stent, the method comprising:
receiving a woven expandable framework comprising one or more knitted-filament regions and one or more undulating-filament regions over a mandrel;
applying a coating to the one or more undulating-filament regions; and
applying a circumferential sleeve around the one or more knitted-filament regions;
wherein the step of applying the coating is performed separately from the step of applying the circumferential sleeve.

19. The method of claim 18, wherein applying a coating to the one or more undulating-filament regions further comprises applying a spray or dip coating.

20. The method of claim 18, wherein applying a circumferential sleeve around the one or more knitted-filament regions further comprises applying one or more of a sheath, a sleeve and a wrap.
